(19) United States
(12) Patent Application Publication
(10) Pub. No.: US 2025/0331927 A1
(43) Pub. Date: Oct. 30, 2025

(54) SYSTEM FOR SURGICAL INSTRUMENT VERIFICATION

(71) Applicants: Smith & Nephew, Inc., Memphis, TN (US); Smith & Nephew Orthopaedics AG, Zug (CH)

(72) Inventors: Eric Ashuckian, Pittsburgh, PA (US); Samuel C. Dumpe, Sewickley, PA (US); Kevin W. Belew, Hernando, MS (US)

(73) Assignees: Smith & Nephew, Inc., Memphis, TN (US); Smith & Nephew Orthopaedics AG, Zug (CH)

(21) Appl. No.: 19/093,560

(22) Filed: Mar. 28, 2025

Related U.S. Application Data
(60) Provisional application No. 63/637,977, filed on Apr. 24, 2024.

(51) Int. Cl. A61B 34/20; A61B 17/00; A61B 17/92; A61F 2/46

(52) U.S. Cl. CPC A61B 34/20; A61B 17/92; A61F 2/4609; A61B 2017/00725; A61B 2017/00738; A61B 2034/2072

(57) ABSTRACT

An orthopedic surgical instrument has an instrument base, a shaft, and an end effector. The shaft is attached to the instrument base such that a distal end of the shaft extends away from the instrument base. The end effector is removably attached to the distal end of the shaft. An implant device can be removably attached to the distal end of the shaft. The end effector has one or more calibration features on an outer surface thereof, the one or more calibration features being provided for position registration and/or verification of the end effector.

### CROSS-REFERENCE TO RELATED APPLICATIONS

This application is a non-provisional of, and claims the benefit of the filing date of, U.S. provisional patent application No. 63/637,977, which was filed Apr. 24, 2024, the entire contents of which are incorporated herein.

### FIELD OF THE DISCLOSURE

The present disclosure relates generally to orthopedic surgical instruments. More particularly, but not exclusively, the present disclosure relates to a system for verifying the proper positioning of such orthopedic surgical instruments, such as during a surgical procedure.

### BACKGROUND OF THE DISCLOSURE

Orthopedic fixation devices (implants) may be used, for example, to stabilize an injury, to support a bone fracture, to fuse a joint, and/or to correct a deformity. Orthopedic fixation devices may be attached permanently or temporarily, and may be attached to the bone at various locations, including implanted within a canal or other cavity of the bone. Some orthopedic fixation devices allow the position and/or orientation of two or more bone pieces, or two or more bones, to be adjusted relative to one another. Orthopedic fixation devices are generally machined or molded from isotropic materials, such as metals including, for example, titanium, titanium alloys, stainless steel, cobalt-chromium alloys, and tantalum.

It is necessary to use orthopedic surgical instruments during any of preparing an implant site, orthopedic implant device trialing, and implantation of a properly-sized orthopedic implant device. However, during any of these procedures, it is necessary to ensure that the orthopedic surgical instruments being used are properly positioned relative to the anatomical structures of the patient on whom the orthopedic surgical procedure is being performed, so that the implantation site and orientation of the orthopedic implant device are anatomically compatible with the patient's physiology and anatomy, including taking into account any unique physiological or anatomical conditions of the patient. It would be beneficial to provide optical or electromagnetic tracking for an offset reamer handle to facilitate navigated or robotic-assisted surgical procedures. When tracking instruments in a navigated or robotic-assisted surgical procedure, particularly when using optical tracking, (1) the tracking array must be visible to the camera and (2) the transformation between the tracking array and the point-of-interest must be known.

In optically tracked navigated and robotically controlled instruments, presently known orthopedic surgical instruments often require divots or other features (e.g., markings or other structures) to verify or calibrate the geometry and positional accuracy of the orthopedic surgical instrument prior to its use. It is advantageous for these divots or other features to be easily accessible by the clinician (e.g., surgeon, surgical technician, surgical representative, etc.), to be easy and quick to interface with, to require minimal to no additional hardware or calibration equipment, measure physiologically relevant features of the instrument, and have a low manufacturing cost. Such features can be used to ensure the geometry of an instrument is not broken or deformed, can be used to verify an assembly is properly and fully assembled, or can be used to calibrate the position of a tool, such as if manufacturing tolerances are significant or if multiple assembled positions are possible.

It is with respect to these and other considerations that the present disclosure may be useful.

### SUMMARY

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

Disclosed herein is an orthopedic surgical instrument including an instrument base, a shaft attached to the instrument base such that a distal end of the shaft extends away from the instrument base, and an end effector configured for removable attachment to the distal end of the shaft. The distal end is configured to have an implant device removably attached thereto, after the end effector. The end effector includes one or more calibration features on an outer surface thereof, the one or more calibration features being configured to provide position registration and/or verification of the end effector.

In any of the preceding or subsequent examples, the one or more calibration features is a groove that extends continuously and concentrically around the outer surface of the end effector.

In any of the preceding or subsequent examples, the groove has a V-shaped profile.

In any of the preceding or subsequent examples, the groove is formed at a midpoint of the end effector along a direction of a longitudinal axis of the shaft.

In any of the preceding or subsequent examples, the one or more calibration features is a series of divots or protrusions that extend uniformly and concentrically around the outer surface of the end effector.

In any of the preceding or subsequent examples, the series of divots or protrusions are formed at a midpoint of the end effector along a direction of a longitudinal axis of the shaft.

In any of the preceding or subsequent examples, the distal end of the shaft includes a threaded portion, such that the end effector and the implant device are configured to thread onto and off of the threaded portion of the shaft.

In any of the preceding or subsequent examples, the implant device is configured to directly contact the end effector.

In any of the preceding or subsequent examples, the shaft is a linear shaft.

In any of the preceding or subsequent examples, the shaft is an offset shaft.

In any of the preceding or subsequent examples, the orthopedic surgical instrument is a cup impactor.

In any of the preceding or subsequent examples, the implant device is an implant cup.

In any of the preceding or subsequent examples, the implant cup is an acetabular cup.

In any of the preceding or subsequent examples, the end effector is formed from a plastic material.

In any of the preceding or subsequent examples, the plastic material includes polyphenylsulfone (PPSU) plastic.

In any of the preceding or subsequent examples, the plastic material consists of polyphenylsulfone (PPSU) plastic.

Examples of the present disclosure provide numerous advantages. In one non-limiting example advantage, a user of the systems disclosed herein can interchangeably use any of a variety of compatible components (e.g., shafts) for the orthopedic surgical instrument. In another non-limiting example advantage, the position of the component (e.g., impactor shell tip) of the orthopedic surgical instrument in/on which the calibration feature(s) is/are provided can be verified independent of the relative positions of the impactor shell tip and the component (e.g., impactor shaft) to which the impactor shell tip is attached. Thus, the impactor shell tip can be easily attached to the impactor shaft without requiring any specific degree of precision as to the engagement of the impactor shell tip with the impactor shaft is not required, since the position of the impactor shell tip can itself be verified independent of the impactor shaft.

Further features and advantages of at least some of the examples of the present invention, as well as the structure and operation of various examples of the present invention, are described in detail below with reference to the accompanying drawings.

### BRIEF DESCRIPTION OF THE DRAWINGS

By way of example, specific examples of the disclosed device will now be described, with reference to the accompanying drawings, in which:

FIG. 1 shows features of a first example of an orthopedic surgical instrument, in the form of a cup impactor.

FIG. 2A shows features of an example impactor shell tip, which is shown in FIG. 1 attached at a distal end of the shaft of the cup impactor.

FIG. 2B shows features of another example impactor shell tip, in which the concentric groove or notch shown on the impactor shell tip in FIG. 2A is replaced with a series of concentrically arranged repeating divots, recesses, or other structures.

FIG. 2C shows features of another example impactor shell tip, in which the repeating divots, recesses, or other structures are provided discretely (e.g., non-continuously) around the outer circumferential surface of the impactor shell tip.

FIG. 3 shows features of a second example of orthopedic surgical instrument, in the form of a cup impactor with an offset shaft.

FIG. 4 is a perspective view of a portion of the offset shaft of the cup impactor shown in FIG. 3.

FIG. 5 is a side view of a portion of the offset shaft of the cup impactor shown in FIGS. 3 and 4.

FIG. 6 is a side cross-sectional view of the shaft and impactor shell tip of the cup impactor shown in FIGS. 1 and 2A, with a cup threaded onto the distal end of the shaft to engage against the impactor shell tip.

FIG. 7 is a side view of an offset impactor with a second example of a calibration fiducial attached thereto.

FIG. 8 is a perspective view of the offset impactor of FIG. 7.

FIG. 9 is a side view of the offset impactor of FIGS. 7 and 8, with the calibration fiducial removed.

FIG. 10 is a side view of an impactor with the second example of a calibration fiducial attached thereto.

FIG. 11 is a perspective view of the impactor of FIG. 10.

FIG. 12 is a side view of the impactor of FIGS. 10 and 11, with the calibration fiducial removed.

The drawings are not necessarily to scale. The drawings are merely representations, not intended to portray specific parameters of the disclosure. The drawings are intended to depict various examples of the disclosure, and therefore are not considered as limiting in scope. In the drawings, like numbering represents like elements.

### DETAILED DESCRIPTION

The subject matter described herein relates generally to orthopedic surgical instruments, such as cup impactors, having fiducial markings formed thereon for position tracking during an orthopedic surgical procedure. Various features or the like of example orthopedic surgical instruments will now be described more fully herein with reference to the accompanying drawings, in which one or more features thereof with a position tracking system will be shown and described. It should be appreciated that the various features may be used independently of, or in combination, with each other. Furthermore, the example orthopedic surgical instruments disclosed herein may be embodied in many different forms and may selectively include one or more concepts, features, or functions described herein. As such, the example cup impactors with the position tracking system, as shown and described herein, should not be construed as being limited to the specific examples set forth herein. Rather, these examples are provided so that this disclosure will convey certain features to those skilled in the art.

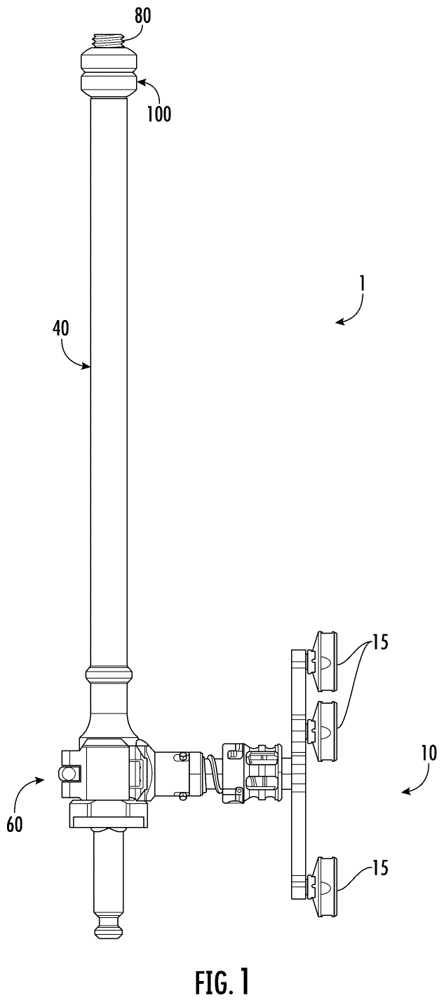

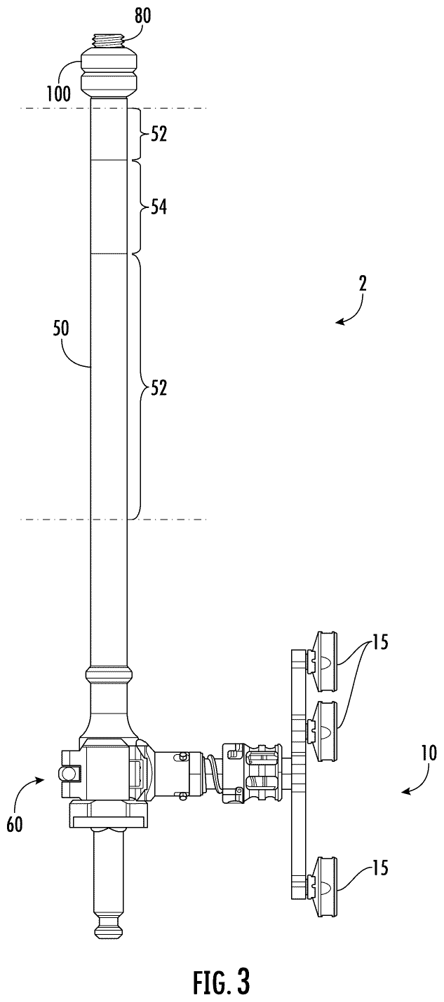

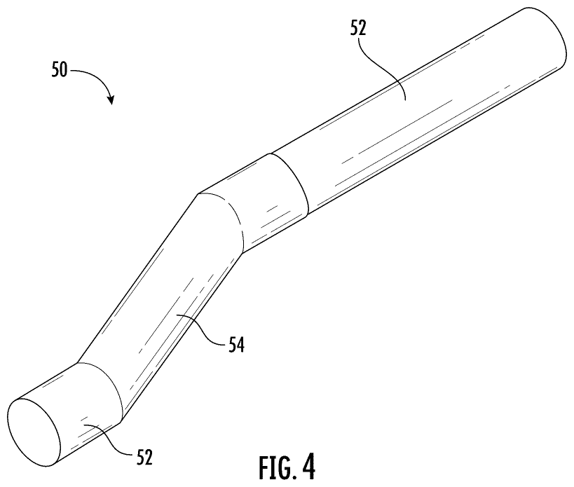

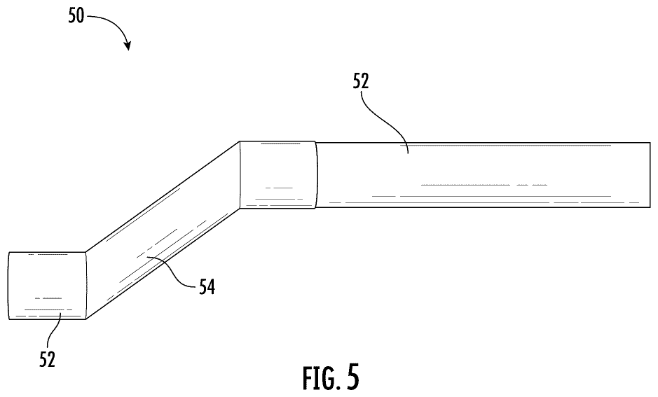

In accordance with one or more features of the present disclosure, an optical or electromagnetic tracking assembly 10 for an orthopedic surgical instrument 1 is disclosed. In the examples disclosed herein, the orthopedic surgical instruments 1 are cup impactors. The orthopedic surgical instrument 1 has a shaft 40, 50 and an impactor shell tip 100 that can be assembled onto the working, or distal end, of the shaft 40, 50. The orthopedic surgical instrument 1 can have a linear shaft 40 (see FIG. 1) or can have an offset shaft 50 (see FIGS. 3-5). The implant device 30 (e.g., an acetabular cup) is coupled to the shaft 40, 50 and the impactor shell tip 100, such as by threading the implant device 30 onto the threaded portion 80 of the shaft 40, 50 to contact (e.g., directly) the impactor shell tip 100. In the examples disclosed herein, the orthopedic surgical instrument 1 is shown having an optical target array 10 that fixedly couples with the handle or shaft 40, 50 in one or more positions at which a translation between the optical target array 10 and the impactor shell tip 100 is known. The translation may refer to coordinates in three-dimensional space such as Cartesian coordinates although such a translation is not limited to any particular coordinate system. In such examples, for instance, an axis for rotation of the optical target array 10 is coaxial with the axis of the shaft 40, 50 or of the impactor shell tip 100. In alternate examples, the optical or electromagnetic tracking array 10 may have a limited number of positions of attachment that each have known translations between the optical target array 10 and the impactor shell tip 100. Note that while the present disclosure illustrates a limited number of different orthopedic surgical instruments 1, namely in the form of cup impactors, the optical or electromagnetic tracking system assemblies, or target arrays 10, are not limited to the specific examples illustrated herein.

That is, in accordance with one or more features of the present disclosure, as illustrated, the orthopedic surgical instrument 1 includes a shaft 40, 50, which includes a first or proximal end and a second or distal (working) end. In use, the first or proximal end is arranged and configured to be engaged by or struck by a surgical impactor or a surgical tool, for example, a hammer, mallet, or other suitable tool for imparting a force to the first or proximal end of the shaft 40, 50. The second or distal working end of the shaft 40, 50 is configured to engage, interact with, etc., an orthopedic device, such as an impactor shell tip 100 that is configured to engage with a surgical implant 30, such as an acetabular cup. When struck at the first or proximal end, the shaft 40, 50 is configured to transmit the force to the impactor shell tip 100 at and/or through the second or distal working end of the shaft 40, 50. In addition, as illustrated, an optical tracker array 10 is coupled to, associated with, etc., the shaft 40, 50 along a length thereof (e.g., between the first or proximal end and the second or distal working end).

As illustrated, in some examples, the distal working end of the shaft 40, 50 includes an externally threaded end portion 80. In use, the impactor shell tip 100 includes an internally threaded bore or cavity 140 for threadably engaging the external threads formed on the distal working end of the shaft 40, 50, although this is but one configuration and the impactor shell tip 100 may be coupled to the shaft 40, 50 by other now known or hereafter developed attachment and/or retention mechanisms. In the illustrated example, the external threads of the threaded portion 80, formed on the distal working end of the shaft 40, 50, extend distally past or beyond (e.g., in the axial or longitudinal direction of the shaft 40, 50) the impactor shell tip 100 after the impactor shell tip 100 is threadably engaged with the external threads on the distal working end of the shaft 40, 50. In other words, the length of the threaded portion 80 of the distal working end of the shaft 40, 50 is greater than the length of the impactor shell tip 100, both lengths being defined in the axial or longitudinal direction of the shaft 40, 50. Thus arranged, the surgical implant 30 (e.g., in the form of an acetabular cup) can be threadably engaged onto the remaining portion of the threaded portion on the distal working end of the shaft 40, 50 that protrude beyond the impactor shell tip 100 after the impactor shell tip 100 is threadably engaged with the external threads of the threaded portion at the distal working end of the shaft 40, 50.

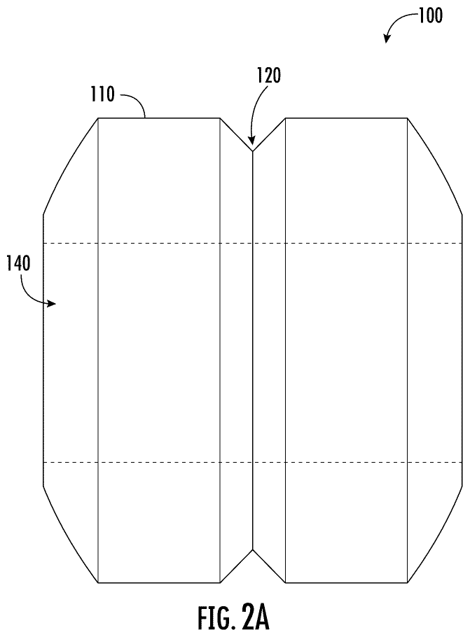

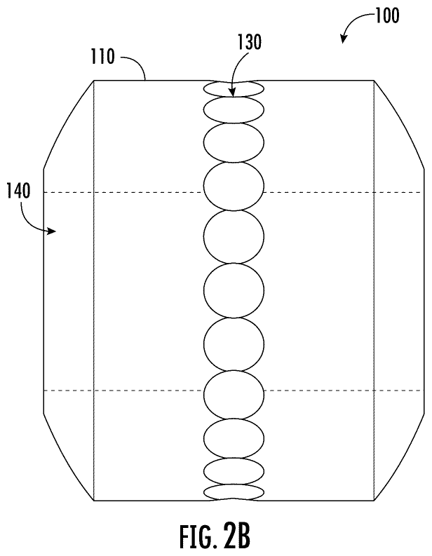

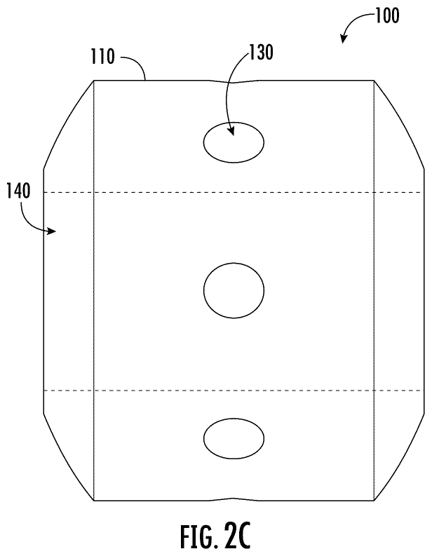

Once assembled, a pointer or position probe can be placed against the registration feature (e.g., groove 120, see FIG. 2A, or array of divots 130, see FIGS. 2B, 2C) formed on the outer surface of the body 110 of the impactor shell tip 100, thereby calibrating the position of the impactor shell tip 100 and, hence, the surgical implant 30 that is positioned directly against (or a prescribed distance away from) the impactor shell tip 100.

Thus arranged, in use, the impactor shell tip 100 can be selectively coupled to and decoupled from the shaft 40, 50, such as by threaded engagement with the external threads formed on the threaded portion 80, at the distal working end of the shaft 40, 50. In use, this enables surgeons to selectively use any of a variety of different shaft 40, 50 such as, for example, shorter length shafts, longer length shafts, straight shafts, offset shafts, etc. Once selected, the appropriate or desired shaft 40, 50 is incorporated into the system and the impactor shell tip 100 is coupled thereto by, for example, threadably engaging the impactor shell tip 100 onto the distal working end of the shaft 40, 50. Next, the surgical implant 30 such as, for example, the acetabular cup, is coupled to the remaining threaded end portion of the shaft 40, 50. Finally, the registration features 120, 130 formed on the outer surface of the body 110 of the impactor shell tip 100 can be located and the position of the impactor shell tip 100 can be verified, such as, for example, by using a pointer or position probe that enables the system to register the precise position of the surgical implant 30 regardless of the shaft 40, 50 to which the impactor shell tip 100 and surgical implant 30 are attached.

Recent developments in navigated surgery and robotic surgery techniques have led to interest in monitoring the progression of surgery and assisting in navigation of surgical instruments during surgery, including positioning and orientation of surgical tools with respect to, for example, a bone. For orthopedic surgical instruments 1 such as a cup impactor, a target array 10 (e.g., optical, electromagnetic, etc.) may be attached (e.g., fixedly, such as in an immobile manner) to the orthopedic surgical instrument 1, adjacent to the proximal, or non-working, end of the shaft 40, 50, to monitor the position of the orthopedic surgical instrument 1 optically. To implement optical position tracking, an unobstructed line-of-sight view of an optical target array 10 must be provided for an optical tracker 10 (e.g., one or more cameras coupled with a computer system) on an orthopedic surgical instrument 1 so the position of, e.g., the impactor shell tip 100, is known to the computer system. The computer system may include memory with code (or instructions), wherein the code, when executed by a processor of the computer system, causes the processor to access to a translation between the optical targets 15 on an optical target array 10 and the impactor shell tip 100 and may determine a location of the impactor shell tip 100 at the distal end of the shaft 40, 50 of the orthopedic surgical instrument 1 based on the position of the optical targets 15 and the detected translatory movements.

Typically, for positional registration or verification, optically tracked tools have a fiducial marking or indentation, commonly referred to as a “divot,” to accurately locate one or more degrees of freedom. Some orthopedic surgical tools, such as cup impactors, only require selective calibration of depth and axis angle for the shaft 40, 50 thereof and, thus, the position tracking thereof can be simplified over known position tracking systems for such orthopedic surgical instruments having components with axisymmetric designs. An array of concentrically arranged divots (130, see FIGS. 2B, 2C) or a continuous groove (120, see FIG. 2A) can be used to selectively locate one or more degrees of freedom of movement of the orthopedic surgical instrument 1. The location of the orthopedic surgical instrument 1 is referred to as being “selectively” verified because movement in degrees of freedom that do not affect use of the orthopedic surgical instrument 1 can be ignored. For the example orthopedic surgical instrument 1 of a cup impactor, a degree of freedom that can be ignored is rotation of the shaft 40, 50 about the longitudinal axis thereof. For example, the position of the central axis is known for the cup impactor of FIG. 1 due to the rigid attachment of the shaft 40, 50 to the base 60 of the instrument 1, however, the position of the impactor shell tip 100 is not precisely known merely by the threaded engagement of the impactor shell tip 100 onto the distal end of the shaft 40, 50. For example, the length and geometry of the shaft 40, 50 of the orthopedic surgical tool 1 may not be precisely known by the system.

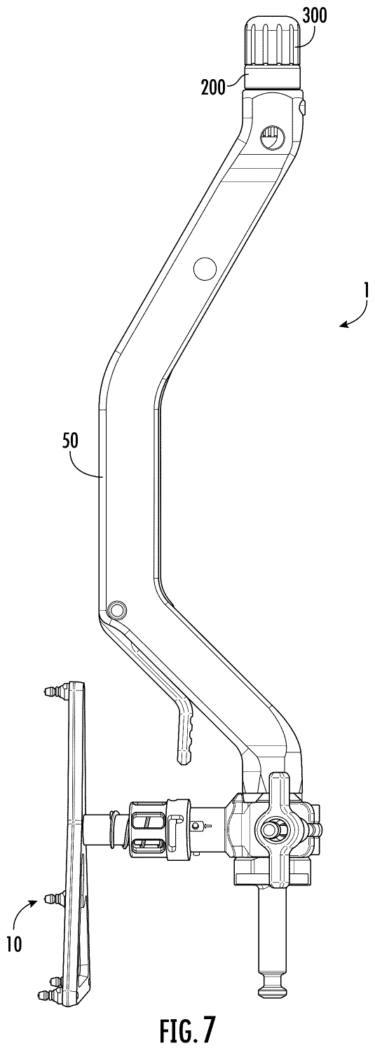

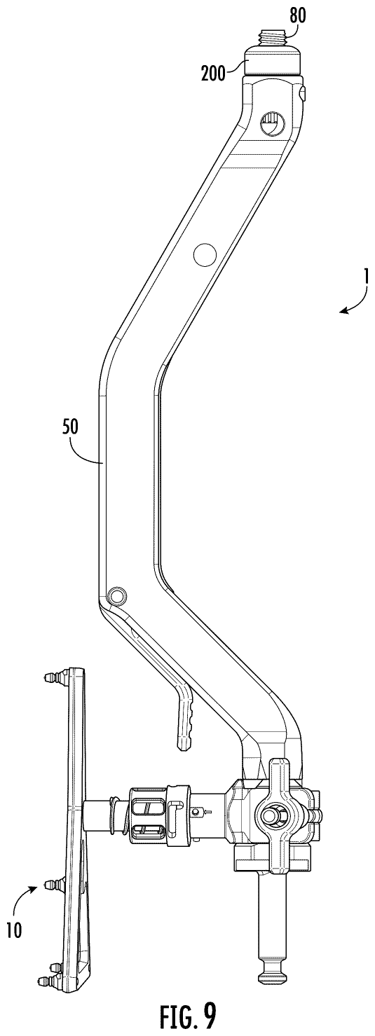

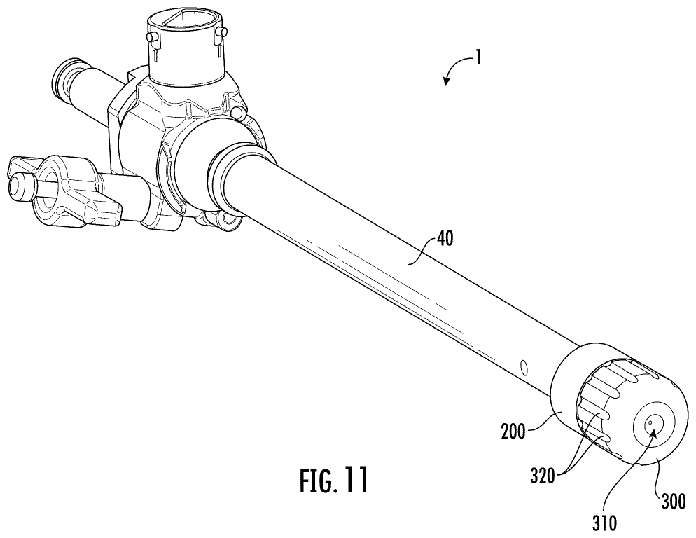

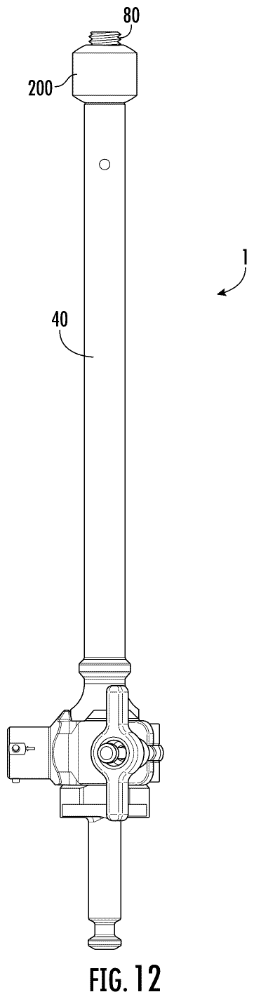

In order to provide this selective positional registration and/or verification of the orthopedic surgical instrument 1, the impactor shell tip 100 has a concentric, continuous groove 120 or other concentric features (e.g., divots or recesses 130) can be added to one or more axisymmetric components thereof. Having the groove 120 (see, e.g., FIG. 2A) or series of divots 130 (see, e.g., FIGS. 2B, 2C) formed concentrically around the impactor shell tip 100 allows the system to precisely determine the position of the impactor shell tip 100 along the longitudinal axis of the shaft 40, 50 so that, accordingly, the position of placement of the surgical implant 30 within the patient can also be precisely determined. In some examples, positional calibration of the orthopedic surgical instrument 1 can be accomplished by inserting or otherwise contacting one calibration feature (see, e.g., calibration head 300, FIGS. 7-12) or three of the divots or recesses 130 (see, e.g., FIGS. 2B, 2C).

The use of a concentric calibration feature, such as the groove 120, is advantageous in some respects over a series of discretely formed calibration features, such as the divots or recesses 130, because such concentric calibration features are easier to precisely manufacture and also easier for the clinician to access. The calibration feature(s) is/are formed on the outer surface of the body 110 of the impactor shell tip 100 to also define the distance between the calibration feature and the surgical implant 30, since the surgical implant 130 is in contact (e.g., direct contact) with the distal end of the impactor shell tip 100. The distance between the distal end of the impactor shell tip and the calibration feature is a prescribed, fixed value. Such calibration features can be provided at a prescribed (i.e., predefined) position on any axisymmetric component, including directly on/in the shaft of the cup impactor shown in FIG. 1. The cup impactor shown in FIG. 3, however, does not have an axisymmetric shaft and, as such, the offset shaft of the cup impactor of FIG. 3 is not a suitable structure onto which such a calibration feature described herein can be provided. As such, in the cup impactor of FIG. 3, the calibration feature is provided on the impactor shell tip, which is the same as the impactor shell tip shown in FIG. 2A.

Thus, in the example cup impactors shown in FIGS. 1 and 3, two position tracking systems are provided. The optical target array 10 can be omitted from the example orthopedic surgical instrument 1, if desired. The working or distal end of the shaft 40, 50 has a selectively couplable interface (e.g., a helically threaded portion 80). The impactor shell tip 100 can threadably engage with the threaded portion 80 of this working or distal end of the shaft 40, 50 in a removable manner (e.g., by being threaded onto and off of the threaded portion 80 of the shaft 40, 50). The impactor shell tip 100 has a calibration feature (e.g., groove 120 or divots 130) formed on an outer surface of the body 110 thereof. In the examples disclosed herein and shown in relation to FIGS. 1-6, in which the orthopedic surgical instrument 1 is a cup impactor, the impactor shell tip 100 has the calibration feature 120, 130 formed at a midpoint in the direction of the longitudinal axis of the shaft 40, 50 onto which the impactor shell tip 100 is threadably engaged. In the example shown in FIG. 2A, the calibration feature is a V-shaped groove 120 that is symmetric and concentric around the body 110 of the impactor shell tip 100, so as to extend continuously around the body 110 of the impactor shell tip. The impactor shell tip 100 can be made of any suitable material, such as PPSU (polyphenylsulfone) plastic.

It is advantageous in some respects for the calibration feature to be formed as a concentric groove 120 instead of the divots 130 shown in FIGS. 2B, 2C because a groove structure is easy for a clinician to access since it is visible and accessible at a radial approach. Having the calibration feature in the shape of a concentric groove 120 is further advantageous over the divots 130 in some respects because such structures are readily capable of being accurately manufactured. For example, the groove 120 can be formed as a single turned feature on a turned impactor shell tip 100. Advantageously, forming a concentric groove 120 is easier on a plastic impactor shell tip 100 than on a large metal instrument, or even a component thereof.

Furthermore, forming the calibration features 120, 130 on a removable component of the orthopedic surgical instrument 1, such as the impactor shell tip 100, is advantageous because this removable component can be easily serviced, replaced (e.g., to replace a worn or used component or even with a redesigned component), etc. with minimal cost. The axisymmetric nature of the concentric groove 120 calibration feature on the structure (e.g., the impactor shell tip 100) that contacts the implant device 30 (e.g., the acetabular cup) allows for accurate position measurement even if the position of the implant device 30 on the impactor shell tip 100 is adjusted, such as may be needed to align holes in the implant device 30 with patient anatomy. Indeed, forming the calibration feature on the body of the impactor shell tip 100 in the form of a concentric groove 120 that is at a longitudinal midpoint of the body 110 of the impactor shell tip 100 is further advantageous because this allows for accurate position measurement even if the impactor shell tip 100 is assembled onto the shaft 40, 50 backwards.

A specific advantage of the presently disclosed subject matter is that it is not necessary to provide a custom-designed orthopedic surgical instrument 1 for use with a robotic surgical system. Instead, the impactor shell tip 100 can be replaced on, for example, a cup impactor, thereby minimizing the components needed for the cup impactor.

The impactor shell tip 100 is the component of orthopedic surgical instrument 1 (e.g., the cup impactor) that contacts (e.g., directly) the implant device 30 (e.g., the implant cup). The implant device 30 is threaded onto the threaded portion 80 of the shaft 40, 50 until it contacts (e.g., directly) the impactor shell tip 100. The proximal end of the shaft 40, 50 (e.g., the end opposite the threaded portion 80) can be struck by a clinician when the cup impactor is determined to be in the anatomically correct position to drive the implant cup into the implant site.

In any of the examples described herein, the concentric groove 120 can be replaced with a series of discrete divots 130 that are concentrically arranged around the structure whose position is being measured, for example, the shaft 40, 50 and/or the impactor shell tip 100. Examples of an impactor shell tip 100 having such an example series of discrete divots 130 concentrically arranged around the outer surface of the impactor shell tip 100 is shown in FIGS. 2B, 2C. The divots 130 shown in FIGS. 2B, 2C can be of any suitable or prescribed shape and/or depth. In some examples, the divots 130 shown in FIGS. 2B, 2C can be protrusions that extend above the outer surface of the body 110 of the impactor shell tip 100, rather than being recessed into the outer surface of the body 110 of the impactor shell tip 100. The system can be configured to detect and issue a warning to a user when a same divot 130 is selected more than once by the user during a positional calibration sequence. The orthopedic surgical instrument 1 can be assembled with any of a plurality of shaft 40, 50 having different lengths based on various criteria, for example, short or long impactor shafts and/or straight or offset impactor shafts 40, 50.

The impactor shell tip 100 is merely an example of an end effector for an orthopedic surgical instrument 1 that can be provided with one or more calibration features 120, 130 for positional registration and verification. The term “end effector” encompasses all such similar devices.

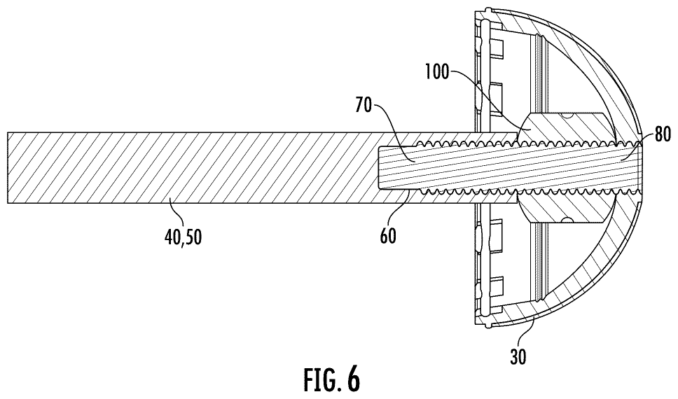

FIG. 6 is a cross-sectional view that shows features of a portion of the example orthopedic surgical instrument 1 shown in FIG. 1, particularly at the distal end of the shaft 40, 50, where the impactor shell tip 100 is threadably engaged onto the threaded portion 80 at the distal, working end of the shaft 40, 50. In this example, the shaft 40, 50 has an internally-threaded cavity 60 that is configured to receive therein, in a threaded manner, a threaded insert 70. The use of the threaded insert 70 is in some respects advantageous because it allows for threaded inserts 70 of any suitable length to be assembled with the shaft 40, 50 depending on, for example, the geometry of the impactor shell tip 100 and the implant device 30. The threaded insert 70 thus has a portion that is contained within the internally-threaded cavity 60 of the shaft 40, 50 and a threaded portion 80 that extends out from and beyond the distal end of the shaft 40, 50. In some examples, the threaded insert 70 and the shaft 40, 50 may have a unitary, or monolithic construction, and can thus be formed as a single piece. As shown in FIG. 6, the impactor shell tip 100 is threaded onto the distally-extending threaded portion 80 of the threaded insert 70. In this example, the impactor shell tip 100 is threaded onto the threaded portion 80 of the threaded insert 70 until the impactor shell tip 100 contacts the distal end of the shaft 40, 50. FIG. 6 further shows that the implant device 30 is threadably engaged over the distally-extending threaded portion 80 of the threaded insert 70 and, specifically, over the part of the threaded portion 80 that extends distally out of and beyond the distal end of the impactor shell tip 100, so that the implant device 30 and the impactor shell tip 100 are in contact (e.g., direct contact) with each other.

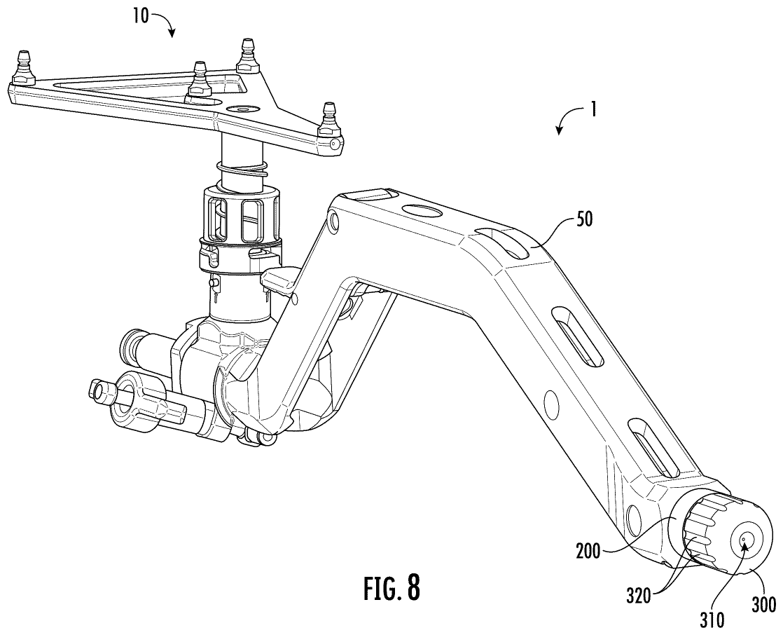

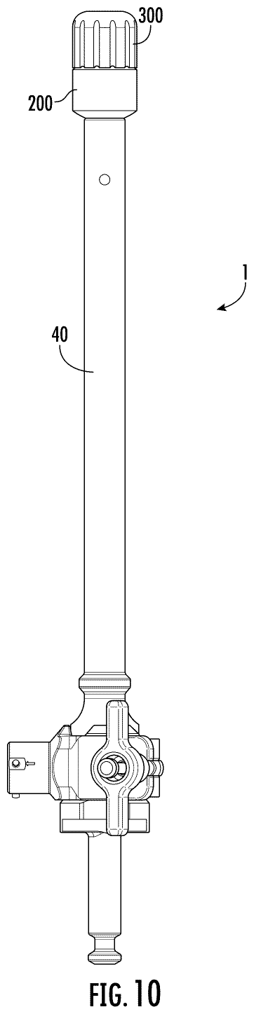

FIGS. 7 and 8 are respective side views of further example orthopedic surgical instruments with a positional calibration fiducial 300 threadably engaged therewith. In FIGS. 7-9, the orthopedic surgical instrument 1 is an offset impactor (e.g., having an offset shaft 50). In FIGS. 10-12, the orthopedic surgical instrument 1 is an impactor with a straight shaft 40. a threaded portion 80 of the shaft 50. A spacer 200 (also sometimes referred to as a “bumper”) can be threadably engaged onto the threaded portion 80 of the shaft 50 to establish a position of the calibration fiducial 300 relative to the shaft 50 and, thus also, relative to the target array 10. The target array 10 can be an optical or electromagnetic tracking device and is rigidly attached to the shaft 50. The spacer 200 can be made of any suitable material, including, for example, a plastic material. The calibration fiducial 300 comprises a central divot 310, which is coaxial with the longitudinal axis of the calibration fiducial 300 and, thus also, coaxial with the longitudinal axis of the threaded portion 80 of the shaft 50. The coaxial alignment of the central divot 310 allows for positional calibration using only a single divot 310. The calibration fiducial 300 advantageously has, on the outer surface thereof, ribs or other suitable structures to aid a user in threading the calibration fiducial onto and off of the threaded portion 80 of the shaft 50, such as manually (e.g., without the aid of a tool).

During an example method of calibrating either of the orthopedic surgical instruments shown in FIGS. 7-12, the method can comprise threadably engaging the spacer 200 onto the threaded portion 80 of the shaft 40, 50 then threadably engaging the calibration fiducial 300 onto the threaded portion 80 of the shaft 40, 50 (e.g., so that no portion of the threaded portion 80 is exposed), performing a calibration operation using the calibration fiducial 300, unthreading (e.g., removing) the calibration fiducial 300 from the threaded portion 80 of the shaft 40, 50, and then threadably engaging a further end effector (e.g., a cup impactor 30, see FIG. 6) onto the threaded portion 80 of the shaft 40, 50. In this method, the calibration fiducial 300 is threaded onto the threaded portion 80 of the shaft 40, 50 until the calibration fiducial 300 is at a prescribed position (e.g., abutting, in direct contact with) relative to the spacer 200. In this example method, the calibration procedure can comprise touching or otherwise engaging a probe with the central divot 310 of the calibration fiducial 300, thereby establishing a position of the calibration fiducial 300 and, thus also, the end of the shaft 40, 50, relative to the target array 10.

While the present disclosure refers to certain examples, numerous modifications, alterations, and changes to the described examples are possible without departing from the sphere and scope of the present disclosure. Accordingly, it is intended that the present disclosure not be limited to the described examples, but that it has the full scope defined by the language of the specification, and equivalents thereof, as would be understood by persons having ordinary skill in the art. The discussion of any example is meant only to be explanatory and is not intended to suggest that the scope of the disclosure, including the claims, is limited to these examples. In other words, while illustrative examples of the disclosure have been described in detail herein, it is to be understood that the inventive concepts may be otherwise variously embodied and employed, and that the descriptions of such examples herein are intended to be construed to include such variations, except as limited by the prior art.

The foregoing discussion has been presented for purposes of illustration and description and is not intended to limit the disclosure to the form or forms disclosed herein. For example, various features of the disclosure are grouped together in one or more examples or configurations for the purpose of streamlining the disclosure. However, it should be understood that various features of the certain examples or configurations of the disclosure may be combined in alternate examples, or configurations. Any example or feature of any section, portion, or any other component shown or particularly described in relation to various examples of similar sections, portions, or components herein may be interchangeably applied to any other similar example or feature shown or described herein. Additionally, components with the same name may be the same or different, and one of ordinary skill in the art would understand each component could be modified in a similar fashion or substituted to perform the same function.

As used herein, an element or step recited in the singular and proceeded with the word “a” or “an” should be understood as not excluding plural elements or steps, unless such exclusion is explicitly recited. Furthermore, references to “one example” of the present disclosure are not intended to be interpreted as excluding the existence of additional examples that also incorporate the recited features.

The phrases “at least one,” “one or more,” and “and/or,” as used herein, are open-ended expressions that are both conjunctive and disjunctive in operation. The terms “a” (or “an”), “one or more” and “at least one” can be used interchangeably herein. The phrases “at least one” and “one or more” include the singular (i.e., only one) and the plural (i.e., more than one, or a plurality). All directional references (e.g., proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader's understanding of the present disclosure, and do not create limitations, particularly as to the position, orientation, or use of this disclosure. Connection references (e.g., engaged, attached, coupled, connected, and joined) are to be construed broadly and may include intermediate members between a collection of elements and relative to movement between elements unless otherwise indicated. As such, connection references do not necessarily infer that two elements are directly connected and in fixed relation to each other. All rotational references describe relative movement between the various elements. Identification references (e.g., primary, secondary, first, second, third, fourth, etc.) are not intended to connote importance or priority but are used to distinguish one feature from another. The drawings are for purposes of illustration only and the dimensions, positions, order and relative to sizes reflected in the drawings attached hereto may vary.

We claim:

1. An orthopedic surgical instrument comprising:
an instrument base;
a shaft attached to the instrument base such that a distal end of the shaft extends away from the instrument base; and
an end effector configured for removable attachment to the distal end of the shaft;
wherein the distal end is configured to have an implant device removably attached thereto; and
wherein the end effector comprises one or more calibration features on an outer surface thereof, the one or more calibration features being configured to provide position registration and/or verification of the end effector.

2. The orthopedic surgical instrument of claim 1, wherein the one or more calibration features is a groove that extends continuously and concentrically around the outer surface of the end effector.

3. The orthopedic surgical instrument of claim 2, wherein the groove has a V-shaped profile.

4. The orthopedic surgical instrument of claim 3, wherein the groove is formed at a midpoint of the end effector along a direction of a longitudinal axis of the shaft.

5. The orthopedic surgical instrument of claim 1, wherein the one or more calibration features is a series of divots or protrusions that extend uniformly and concentrically around the outer surface of the end effector.

6. The orthopedic surgical instrument of claim 5, wherein the series of divots or protrusions are formed at a midpoint of the end effector along a direction of a longitudinal axis of the shaft.

7. The orthopedic surgical instrument of claim 1, wherein the distal end of the shaft comprises a threaded portion, such that the end effector and the implant device are configured to thread onto and off of the threaded portion of the shaft.

8. The orthopedic surgical instrument of claim 7, wherein the implant device is configured to directly contact the end effector.

9. The orthopedic surgical instrument of claim 1, wherein the shaft is a linear shaft.

10. The orthopedic surgical instrument of claim 1, wherein the shaft is an offset shaft.

11. The orthopedic surgical instrument of claim 1, wherein the orthopedic surgical instrument is a cup impactor.

12. The orthopedic surgical instrument of claim 11, wherein the implant device is an implant cup.

13. The orthopedic surgical instrument of claim 12, wherein the implant cup is an acetabular cup.

14. The orthopedic surgical instrument of claim 1, wherein the end effector is formed from a plastic material.

15. The orthopedic surgical instrument of claim 14, wherein the plastic material comprises or consists of polyphenylsulfone (PPSU) plastic.

16. The orthopedic surgical instrument of claim 1, wherein the implant device is configured for removable attachment to the distal end of the shaft after the end effector has been attached to the distal end of the shaft.

17. The orthopedic surgical instrument of claim 1, comprising a spacer configured for threading onto the distal end of the shaft before the end effector.

18. The orthopedic surgical instrument of claim 1, wherein the one or more calibration features is a single central divot that is formed on a distal end of the end effector.

19. The orthopedic surgical instrument of claim 18, wherein the single central divot is centered on the distal end of the end effector, such that a central longitudinal axis of the end effector passes through the single central divot.

20. The orthopedic surgical instrument of claim 19, wherein the central longitudinal axis of the end effector is coaxial with a longitudinal axis of a portion of the shaft that defines the distal end of the shaft.
